(19) United States
(12) Patent Application Publication
(10) Pub. No.: US 2016/0082065 A1
(43) Pub. Date: Mar. 24, 2016

(54) PROPHYLACTIC USE OF NEUROPROTECTANTS IN SPORTS-RELATED TRAUMATIC BRAIN INJURY

(71) Applicant: NEUROLOGICS GROUP, LLC, SALT LAKE CITY, UT (US)

(72) Inventors: Kevin James Brown, Klamath Falls, OR (US); Gary Brown, Lehi, UT (US)

(21) Appl. No.: 14/863,258

(22) Filed: Sep. 23, 2015

Related U.S. Application Data
(60) Provisional application No. 62/054,236, filed on Sep. 23, 2014.

(51) Int. Cl. A61K 36/67; A61K 31/355; A61K 31/4525; A61K 31/315; A61K 31/202; A61K 31/385; A61K 31/195; A61K 31/194

(52) U.S. Cl. CPC A61K 36/67; A61K 31/195; A61K 31/355; A61K 31/194; A61K 31/315; A61K 31/202; A61K 31/385; A61K 31/4525

(57) ABSTRACT

Dietary supplements for reducing damage to brain cells characterized by loss of brain cells or loss of brain cell function in patients that have suffered from traumatic brain injury (“TBI”) while participating in sports and/or other athletic events are disclosed herein. The Nutraceutical compositions offer possible ameliorating effects for the secondary phase of TBI. Creatine, vitamin E, zinc, magnesium, DHA, and lipoic acid, taken together, offer a prophylactic measure to ameliorate brain damage that occurs during the secondary phase of SRTBI.

### CROSS-REFERENCE TO RELATED APPLICATIONS

This patent application claims priority to U.S. Provisional Patent Application No. 62/054,236 entitled THE PROPHYLACTIC USE OF NEUROPROTECTANTS IN SPORTS-RELATED TRAUMATIC BRAIN INJURY, filed on Sep. 23, 2014, which is incorporated herein by reference in its entirety.

### TECHNICAL FIELD

The present disclosure relates to the prophylactic use of Nutraceutical supplements for mitigating brain damage, either by loss of brain cells or loss of brain cell function, in patients that sustain traumatic brain injury (“TBI”), for example, while participating in sports and/or other athletic events.

### BACKGROUND

Approximately 1.6 million persons in the United States suffer TBI every year. These injuries result in 1.3 million persons who are treated and released from hospital/clinic emergency departments, 235,000 hospitalizations, and 50,000 deaths. Half of all those suffering TBI will suffer temporary impairment, and 80,000-90,000 will suffer permanent disability. Of all TBI sufferers in the United States, approximately 300,000 experience their injuries while participating in sports or other athletic events.

One of the tragedies associated with sports-related traumatic brain injury (“SRTBI”) is the severe injury and death of previously healthy individuals, including many young adults. SRTBI often goes unreported and untreated and can have cumulative effects. Participants in contact sports such as, for example, boxing, hockey, soccer, basketball, rugby, and football are at risk of repeated concussions, which may result in subdural hematomas, permanent loss of cognitive function, temporary or permanent disability, or death.

Despite the predictability and prevalence of SRTBI and the devastating consequences of the associated injuries, there are few ways to treat or prevent SRTBI. Most efforts to reduce SRTBI have focused on rule, training, and equipment changes. Such efforts have only succeeded in reducing SRTBI to the current level. Without fundamentally changing the rules and nature of any given sport or athletic event to eliminate the inherent danger of SRTBI, further efforts of this type will have only a marginal benefit. Furthermore, almost all of the effort spent in reducing SRTBI has been aimed at reducing damage that results from the initial mechanical impact to the brain. In this first stage of a traumatic brain injury, an initial mechanical impact to the brain causes necrotic cell death, which is characterized by the shearing of nerve fibers known as “axons” and the tearing, and laceration of other brain tissues. It seems that this initial trauma is an inescapable consequence of participating in contact sports.

TBI or SRTBI, however, is not a simple, single phase injury. The second phase of TBI/SRTBI begins within a period of minutes to days after the initial mechanical impact. During this second phase, a person can develop further brain damage as the result of chemical, rather than mechanical, processes. In the body's subsequent attempt to recover from the initial mechanical trauma, severely injured brain cells are purged, thereby causing a second and, in some cases, larger wave of brain cell die off. Injured brain cells that have not died via immediate necrotic cell death begin an adaptive response by repairing their injured elements. Brain cells that the body perceives as being damaged beyond repair are actively eliminated through a process called “apoptosis,” “programmed cell death,” and “secondary excitotoxic cascade”.

Beyond rule, training and equipment changes, little more can be done to limit or prevent the primary trauma of SRTBI. Furthermore, most research up to this point has focused on the development of a post-SRTBI treatment for use by medical practitioners. Very little attention has been paid to ameliorating the secondary phase injury of SRTBI, that of apoptosis or programmed cell death. At this time, the secondary phase of SRTBI presents the best possibility for reducing injury from SRTBI. Because programmed cell death in the brain can be influenced by the chemical and physiological properties of the brain, it is treatable; whereas the necrotic cell death caused by the initial mechanical impact of SRTBI is not.

Current research in TBI suggests that neuroprotective agents might be used prophylactically to ameliorate the effects of SRTBI. Among the neuroprotectants that are candidates for such a use are drugs such as Cyclosporin A, a powerful immunosuppressive drug, and Erythropoietin, a glycoprotein, which is used to treat the anemia associated with renal failure, HIV, and cancer. Progesterone and estrogen also have been found to have some neuroprotective properties. Unfortunately, these substances can produce significant side-affects and cannot be used generally by those at risk for SRTBI. There is, however, a class of Nutraceutical dietary supplements that can be used widely by those participating in contact sports, with little or no side effects. These dietary supplements include, but are not limited to, creatine compounds, vitamin E compounds, magnesium compounds, zinc compounds, docosahexaenoic acid (“DHA”) compounds, and lipoic acid compounds. Taken together, these supplements offer a prophylactic treatment to ameliorate injury that occurs during the secondary phase of SRTBI.

### SUMMARY

The present disclosure provides safe and effective methods of ameliorating the damage that occurs during the second phase of SRTBI in a patient at risk thereof by administering a prophylactic, or preventive, therapeutically effective amount of the Nutraceuticals described herein. The combination of Nutraceuticals described herein provides unexpected beneficial results in the prophylactic treatment of one or more symptoms associated with SRTBI.

Accordingly, the present disclosure provides a Nutraceutical formulation for treating apoptosis, programmed cell death, and other forms of brain damage associated with the second stage of SRTBI. In some embodiments, the formulation comprises one or more creatine compounds, one or more vitamin E compounds, one or more magnesium compounds, one or more zinc compounds, one or more DHA compounds, one or more lipoic acid compounds, or any combination thereof.

In one embodiment of the present disclosure, the Nutraceutical formulation comprises one or more creatine compounds. Illustrative creatine compounds include, but are not limited to, creatine monohydrate, creatine anhydrous, creatine phosphate, creatine malate, creatine tartrate, creatine HMB, creatine ester, effervescent creatine, creatine titrate, or any combination of the foregoing.

In one embodiment of the present disclosure, the Nutraceutical formulation comprises one or more vitamin E compounds. Illustrative vitamin E compounds include, but are not limited to, tocopherols such as alpha-tocopherols, a tocopherol form of alpha-tocopheryl acetate, alpha-tocopheryl succinate, d-alpha tocopheryl, d-alpha tocopheryl, or any combination of the foregoing; or in the tocotrienol form of alpha-, beta-, gamma-, or delta-tocotrienol, or any combination of the foregoing. Combinations of tocopherols and tocotrienols can also be used.

In one embodiment of the present disclosure, the Nutraceutical formulation comprises one or more magnesium compounds. Illustrative magnesium compounds include, but are not limited to, magnesium salts, magnesium oxide, magnesium sulfate, magnesium carbonate, magnesium aspartate, magnesium orotate, magnesium glycinate, magnesium citrate/malate, magnesium lactate, magnesium L-lactate dehydrate, magnesium chloride, magnesium gluceptate, magnesium gluconate, magnesium hydroxide, magnesium pidolate, magnesium citrate tribasic anhydrous, or any combination of the foregoing.

In one embodiment of the present disclosure, the Nutraceutical formulation comprises one or more zinc compounds. Illustrative zinc compounds include, but are not limited to, zinc salts, zinc oxide, zinc sulfate, zinc-histidine, zinc-methionine, zinc-cysteine, zinc picolinate, zinc orotate, zinc gluconate, zinc monomethionine, zinc glycinate, zinc lactate dihydrate, or any combination of the foregoing.

In one embodiment of the present disclosure, the Nutraceutical formulation comprises one or more DHA compounds.

In one embodiment of the present disclosure, the Nutraceutical formulation comprises one or more lipoic acid compounds. Lipoic acid can also be known as alpha lipoic acid, or α-lipoic acid.

### DETAILED DESCRIPTION

It is to be understood that the terminology used herein is for the purpose of describing particular embodiments only, and is not intended to limit the scope of the present disclosure which will be limited only by the appended claims.

“Bioperine” is an extract of piper nigrum L or piper longum that contains piperine. For example, Bioperine can contain about 95 percent piperine.

“Creatine” refers to and includes any nitrogenous acid in the form of C₄H₉N₃O₂, and any derivation thereof, including, without limitation, the forms of creatine identified above in the Summary.

“DHA” refers to docosahexaenoic acid and any derivatives thereof. DHA includes, without limitation, compounds in the form of C₂₂H₃₂O₂, and any derivation thereof.

“Lipoic acid” refers to alpha lipoic acid (or α-lipoic acid) and any derivatives thereof. Lipoic acid includes, without limitation, compounds in the form of C₈H₁₄O₂S₂, and any derivation thereof.

“Magnesium” refers to and includes the chemical element magnesium, or any derivative thereof, including, without limitation, any of the forms of magnesium identified above in the Summary.

“Nutraceutical” refers to and includes any substance that provides medicinal or health benefits, including the prevention and/or treatment of disease, including, without limitation, creatine, zinc, magnesium, vitamin E, DHA, and lipoic acid. A Nutraceutical may be an isolated nutrient, dietary supplement, genetically engineered food, herbal product, etc. The individual Nutraceuticals of the disclosure can be obtained commercially, or can be made by methods known in the art.

“Patient” refers to humans, including males and females, children and adults.

“SRTBI” refers to a sports-related traumatic brain injury.

“TBI” refers to a traumatic brain injury.

“Vitamin E” refers to and includes any of several fat-soluble vitamins that are chemically tocopherols or tocotrienols, or any derivative thereof, including, without limitation, any of the forms identified above in the Summary.

“Zinc” refers to and includes the chemical element zinc and any of derivatives thereof, including, without limitation, any of the forms identified above in the Summary.

The disclosure provides safe and effective methods of ameliorating the damage that occurs during the second phase of SRTBI by administering a therapeutically effective amount of the Nutraceuticals provided herein prior to the patient's participation in sports or other athletic events that put the patient at risk of SRTBI. Typical symptoms of SRTBI and, in particular, the subsequent apoptosis or programmed cell death include lightheadedness, dizziness, confusion, headaches, blurred vision, ringing in the ears, poor sleep patterns and/or a change in sleeping patterns, behavioral or mood changes, and loss of memory, concentration, and other cognitive function. Moderate or severe SRTBI may include dilation of pupils, chronic headaches, serial vomiting or nausea, loss of motor coordination, difficulty with speech, convulsions, seizures, inability to awaken from sleep, and weakness in the extremities.

The second phase of SRTBI can begin within a period of minutes to days after an initial mechanical trauma to the brain. During this second phase, a patient can develop brain damage beyond that caused by the initial trauma. As the brain recovers from the initial trauma, severely injured brain cells are purged, thereby causing a second and, in some cases, larger wave of brain cell die off. Brain cells that the body perceives as being damaged beyond repair are actively eliminated through a chemical process called “apoptosis” or “programmed cell death”.

A patient at risk of SRTBI can ameliorate the injury associated with the second phase of SRTBI by taking the disclosed Nutraceutical composition, which can comprise one or more of creatine, magnesium, vitamin E, zinc, DHA, and lipoic acid, prior to participating in sports related activities and other athletic events. For example, a diet enhanced by creatine can have many neuroprotective benefits. For example, creatine enhanced diets can provide neuroprotection in cases of spinal cord injury and TBI. Creatine enhanced diets can provide significant protection from synaptic dysfunction and cortical tissue loss in cases of TBI. Athletes who supplement their diet with creatine can be afforded the neuroprotective benefit through the chronic ingestion of creatine. Creatine can also suppress the accumulation of lactic acid and free fatty acids, which are markers for the excitotoxic secondary phase of TBI. If taken prophylactically, creatine can contribute to the maintenance of mitochondrial homeostasis and integrity resulting in a neuroprotective effect. In some embodiments, creatine enhanced diets of 1% creatine monohydrate taken for a minimum of 5 days prior to TBI can provide an amelioration of the effects of TBI.

Vitamin E is important for normal neurological structure and functioning. For example, it is an important lipid-soluble, chain-breaking antioxidant protecting the body and the brain. Tocotrienol forms of Vitamin E, for example, can block glutamate-induced cell death, a contributor in the second phase of TBI, specifically, secondary excitotoxic cascade. Tocotrienol has been found to be neuroprotective in animal testing. Vitamin E also can have a powerful neuroprotective and antioxidant effect. Vitamin E is also safe for regular and continuous consumption by humans. Vitamin E, and particular the tocotrienol forms, can be a safe dietary supplement that can provide significant neuroprotectant benefits that can contribute to ameliorating the effects of SRTBI.

The dietary intake of Omega-3 fatty acids, such as DHA and eicosapentaenoic acid (EPA), can have multiple health benefits. For example, DHA can restore cellular energetics, reduce oxidative stress, and inflammation; repair cellular damage; and mitigate the apoptosis after TBI. DHA supplementation can also lead to the recovery of spatial ability; a reduction in the inflammatory response to injury; improvement in behavioral deficits caused by deficiency; a reduction in oxidative stress, improvement in neuronal function, and improvement in learning ability after TBI; a reduction in axonal injury after TBI; an increase in sensimotor outcome; improvement in glutamatergic synaptic transmission; and a reduction in excitotoxic cell death in primary neurons. Additionally, unlike EPA or Omega-6 fatty acids, DHA can reduce AMPA receptor-mediated cell death. DHA is a primary, and common, fatty acid found in the human brain and represents 97% of the Omega-3 fatty acid content of the brain. DHA depletion can result in increased susceptibility to TBI and impaired recovery. DHA may further serve as a Nutraceutical agent and precondition the brain to make it more resilient to injury. DHA is bioavailable and passes readily through the blood brain barrier when taken as a dietary supplement. Those at significant risk of TBI may be preloaded with DHA to provide protection against the acute effects of TBI. In total, DHA can be a good component for prophylactic neuroprotection since it is well tolerated and easy to administer, it has a long and established record of administration and dosages are established, and it has demonstrated neuroprotective properties.

Lipoic acid, α-lipoic acid, or alpha-lipoic acid, chemically designated 1,2-dithiolane-3-pentanoic acid; 2-dithiolane-3 valeric acid; and 6,8-dithiooctanoic acid, is a naturally occurring substance that is taken up and reduced in cells into dihydrolipoate, a potent antioxidant and neuroprotectant. Alpha-lipoic acid can be converted in the body into dihydrolipoate. However, this process can take at least two hours to occur in the body. Once converted into dihydrolipoate, alpha-lipoic acid can offer neuroprotection in several ways. For example, alpha-lipoic acid can provide neuroprotection through scavenging free radicals, chelating transition metals, anti-inflammatory antioxidation, increasing glutathione, scavenging carbonyls, stimulating glucose uptake, resisting cytotoxicity, and protecting against ethanol damage. Alpha-lipoic acid can also intervene in the free radical or reactive oxygen species (ROS) cycle that is a characteristic of the excitotoxic cascade. Alpha-lipoic acid is a potent antioxidant that can reduce oxidative stress and increase glutathione levels. Another effect of alpha-lipoic acid is that it can enhance the effect of other antioxidants like tocotrienol. Alpha-lipoic acid may also have value in increasing athletic performance by reducing oxidative stress.

Alpha-lipoic acid has very few side effects and is safe for use by healthy individuals. It can be rapidly converted into dihydrolipoate by the body from oral dietary administration. Alpha-lipoic acid is also effective as a neuroprotectant prophylactically. Alpha-lipoic acid can be utilized in a combination therapy with other antioxidants, and because of its ability to chelate transitional metals it can work well in combination therapies with metals. Because of the wide-ranging and synergistic effects of alpha-lipoic acid and the ease and safety of administration can be a good component for prophylactic neuroprotection.

Zinc is important to normal brain functioning and is also important in neuronal repair. In TBI, urinary zinc excretion can be increased (up to 14 times normal) and serum zinc can be depressed, suggesting that zinc deficiency plays a part in secondary brain injury. However, free zinc, the approximately 10% of the zinc present in the body which is not bound to proteins, can contribute to neuronal death in TBI. Zinc is also neuroprotective. Zinc deficiency that develops after TBI can significantly increase neuronal death. Even moderate zinc deficiency can significantly increase both necrotic and apoptotic cell death. More extreme zinc deficiency can lead to increased apoptotic cell death at the site of the injury. Also, in zinc deficient subjects the second phase of cell death can increase to 4 weeks. The dietary administration of post-trauma zinc does not cause increased cell death. Zinc administered previous to TBI can reduce neuronal cell death in both phases of TBI, necrotic and apoptotic, providing neuroprotection. Similar neuroprotection from pretreatment can be found with adequate and supplemental zinc. However, the administration of zinc post injury has little benefit. Zinc supplementation can also be associated with improved recovery rates in patients with severe closed head injuries. Zinc, as a basic nutrient necessary for normal brain functioning that also provides neuroprotection, can be a good component for a neuroprotective prophylactic regime.

Magnesium is a neuroprotectant important for the normal operation of the brain. Magnesium is important for cellular bioenergetics, RNA aggregation, protein synthesis, the functioning of ATPase, and plasma-membrane integrity. In TBI, both serum magnesium and ionized or free magnesium can decrease. In the case of free magnesium, the decrease can be as much as 40-60%. Since magnesium is important for adequate blood flow in the brain, hypomagnesemia can contribute to neuronal death. However, recent studies have called into question the efficacy of hypermagnesemia as a post TBI treatment. Post TBI treatment with magnesium does not improve outcomes in TBI patients. However, magnesium pre-treatment can ameliorate injury induced impairment in both working and reference memory. Magnesium, like the other dietary supplements discussed herein, seems to be most beneficial in cases of TBI if present in the brain in sufficient amounts at the time of injury. However, most research up to this point has been focused on post TBI treatment use by emergency medical practitioners post-injury. While post-injury hyper magnesia should be used with caution, magnesium administered pre-injury can be neuroprotective. Magnesium offers neuroprotection pre-TBI, though not perhaps post-TBI, and can be a good component for a prophylactic TBI regime.

Gaining the neuroprotection available from nutritional supplementation is a complex process. Not only should the right substances be ingested, they should be used in the proper forms and amounts. The bioavailability of the supplements and the proper form and delivery system for maximum uptake should also be considered. The following discussion considers various elements of embodiments of the present disclosure. As discussed below, the present disclosure is not limited to these elements. Other forms, including, without limitation, varying delivery methods, ingredient levels and ratios, ingredient forms, carrier agents, and co-therapies can be used.

Despite the availability of new creatine compounds that claim greater bioavailability, the research on the neuroprotective benefits of creatine supplementation use the compound creatine monohydrate for their studies. The administration of creatine as part of a prophylactic neuroprotective regime is complicated by the difficulty in administering dietary creatine in a way that can be readily absorbed and used by the brain. One problem is that creatine is not water soluble and is also sensitive to acid environments. Creatine can break down into creatinine in acid environments such as the stomach, therefore creatine should be administered in a way that allows for rapid uptake and optimal gastric emptying. Creatine may be administered in solution with a complex of simple carbohydrates to ensure gastric emptying since each form (e.g., sucrose, glucose, fructose, dextrose, maltodextrin, and other polymers) has a different receptor for absorption. In some embodiments, creatine can be consumed in a pH neutral solution of at least 300 milliliters of liquid along with a combination of simple carbohydrates to promote rapid uptake by the body and bioavailability. Another issue with creatine supplementation is ensuring the proper dosage. While some creatine regimens specifically designed for muscular enhancement have a loading phase of up to 20 g per day, research has established that a dose of 3 g is adequate for muscular supplementation. In some embodiments, dosage levels for creatine supplementation for neuroprotection against SRTBI can be slightly higher so as have creatine available for both the muscular demand and the brain.

In the use of vitamin E supplementation for the purpose of neuroprotection, one concern is the type of vitamin E used. Vitamin E is a generic name for eight substances that have vitamin E activity. Four of these eight substances are tocopherols and four are tocotrienols. The difference between the two is that tocotrienols have an isoprenoid tail with three unsaturated points while tocopherols have a saturated phytyl tail. In some embodiments, tocotrienols can be multifold more potent as a neuroprotectant than tocopherols. In obtaining neuroprotective benefits through dietary supplementation, the inclusion of mostly tocotrienols, instead of the more readily available tocopherols, can be desired. Oral supplementation with tocotrienols can reach the brain and offers neuroprotective benefits. Combinations of tocotrienols and tocopherols can also be used.

Dietary supplementation with DHA can have multiple benefits. For example, DHA, like other Omega-3 fatty acids, can possess a wide range of systemic benefits. Dietary supplementation with DHA, usually in combination with EPA, in the form of fish oil, is common for cardiovascular goals. However, the nutraceutic use of DHA alone can have advantages. While the brain needs both DHA and EPA to function, EPA represents less than 1% of the total brain lipids and the body can, and does, convert DHA into EPA. While evidence suggests that EPA may be important for brain efficiency, there is little or no evidence to suggest that it is effective in TBI. However, dietary supplementation with DHA alone can have the advantage of not being dependent on marine sources. DHA can be obtained from algal sources in a controlled laboratory environment, free from contamination or possible pollution. This can reduce or eliminate the worry about procurement, pollution, and purity that have dogged marine sources. Finally, because of the long history of DHA supplementation in humans, the dosage is well known and it has a long history of tolerance in doses up to 4 g per day.

In some embodiments, supplementation with alpha-lipoic acid needs to occur in advance of injury in order for it to be converted into dihydrolipoate. Alpha-lipoic acid can cross the blood-brain barrier and can be bioavailable as dihydrolipoate after about two hours. Alpha-lipoic acid is well tolerated in a dietary regime and, in some embodiments, a dose of about 600 mg a day can be used. Alpha-lipoic acid can be administered with the mineral selenium, as selenium can have an enhancing effect. Alpha-lipoic acid can offer significant protection from oxidative stress and can be a potent neuroprotectant.

Dietary zinc supplementation has neuroprotective qualities when taken prophylactically. For example, zinc deficiency can compound the injury associated with the secondary phase of TBI. Therefore, zinc supplementation can be used to establish adequate zinc levels within the brain. However, zinc like other minerals can have problems with uptake and bioavailability. In some embodiments, Bioperine can aid in the bioavailability of minerals such as zinc. So a supplementation regime that features zinc can also feature an uptake aid such as Bioperine. Another issue with zinc supplementation is the role of zinc the post-TBI. Exposure to synaptically-released zinc may contribute to cell death post-TBI. Though little to nothing can be done to prevent the release of synaptically bound zinc as free zinc post-TBI, zinc can still be an important neuroprotective element. Adequate or slightly supplemental zinc is a goal of supplementation, and dosages that approximate the United States Department of Agriculture (“USDA”) recommended daily amount can be used.

The pre-injury or prophylactic use of magnesium provides neuroprotective benefits, however, for these benefits to be realized it is important for magnesium to be available to the brain. One method that maintains magnesium levels in the brain is consistent dietary supplementation. Like zinc supplementation, the uptake of magnesium by the body can be aided by an uptake aid such as Bioperine. Since adequate or slightly supplemental magnesium is a goal of supplementation, dosages that approximate USDA recommended daily amount can be used.

In one embodiment, the Nutraceutical composition comprises (i) one or more creatine compounds, pro-drugs, metabolites, or derivatives thereof; (ii) one or more vitamin E compounds, pro-drugs, metabolites, or derivatives thereof; (iii) one or more magnesium compounds, pro-drugs, metabolites, or derivatives thereof; (iv) one or more zinc compounds, pro-drugs, metabolites, or derivatives thereof; (v) one or more DHA compounds, pro-drugs, metabolites, or derivatives thereof; and (vi) one or more lipoic acid compounds, pro-drugs, metabolites, or derivatives thereof.

Various amounts of creatine compounds, vitamin E compounds, magnesium compounds, zinc compounds, DHA compounds, and lipoic acid compounds can be used can be used in the Nutraceutical composition. For example, in some embodiments, the amount of the one or more creatine compounds used is from about 2500 mg to about 7000 mg, from about 3000 mg to about 7000 mg, from about 4000 mg to about 6000 mg, or from about 4500 mg to about 5500 mg. In other embodiments, the amount of the one or more creatine compounds used is at least about 2500 mg, at least about 3000 mg, at least about 4000 mg, or at least about 4500 mg.

In some embodiments, the amount of the one or more vitamin E compounds used is from about 25 mg to about 400 mg, from about 25 mg to about 300 mg, from about 25 mg to about 250 mg, from about 25 mg to about 200 mg, from about 35 mg to about 200 mg, or from about 50 mg to about 150 mg. In other embodiments, the amount of the one or more vitamin E compounds used is at least about 25 mg, at least about 35 mg, or at least about 50 mg.

In some embodiments, the amount of the one or more magnesium compounds used is from about 100 mg to about 1000 mg, from about 200 mg to about 800 mg, from about 250 mg to about 650 mg, or from about 300 mg to about 500 mg. In other embodiments, the amount of the one or more magnesium compounds used is at least about 100 mg, at least about 200 mg, at least about 250 mg, or at least about 300 mg. Additional amounts of the one or more magnesium compounds can also be used. For example, in further embodiments, the amount of the one or more magnesium compounds used can be up to about 15 g, about 20 g, about 25 g, about 30 g, or more.

In some embodiments, the amount of the one or more zinc compounds used is from about 5 mg to about 50 mg, from about 5 mg to about 40 mg, from about 7.5 mg to about 40 mg, from about 10 mg to about 35 mg, from about 10 mg to about 20 mg, or from about 12 mg to about 18 mg. In other embodiments, the amount of the one or more zinc compounds used is at least about 5 mg, at least about 7.5 mg, at least about 10 mg, or at least about 12 mg. Additional amounts of the one or more zinc compounds can also be used. For example, in further embodiments, the amount of the one or more zinc compounds used can be up to about 5 g, about 10 g, about 15 g, about 20 g, or more.

In some embodiments, the amount of the one or more DHA compounds used is from about 200 mg to about 2500 mg, from about 500 mg to about 2500 mg, from about 700 mg to about 2000 mg, from about 750 mg to about 1500 mg, from about 800 mg to about 1200 mg, or from about 900 mg to about 1100 mg. In other embodiments, the amount of the one or more DHA compounds used is at least about 200 mg, at least about 500 mg, at least about 700 mg, at least about 750 mg, at least about 800 mg, or at least about 900 mg.

In some embodiments, the amount of the one or more lipoic acid compounds used is from about 100 mg to about 800 mg, from about 150 mg to about 600 mg, from about 200 mg to about 400 mg, or from about 250 mg to about 350 mg. In other embodiments, the amount of the one or more lipoic acid compounds used is at least about 100 mg, at least about 150 mg, at least about 200 mg, or at least about 250 mg.

In further embodiments, the Nutraceutical composition comprises one or more creatine compounds in an amount from about 4000 mg to about 6000 mg; one or more vitamin E compounds in an amount from about 35 mg to about 200 mg; one or more magnesium compounds in an amount from about 250 mg to about 650 mg; one or more zinc compounds in an amount from about 10 mg to about 20 mg; one or more DHA compounds in an amount from about 200 mg to about 1200 mg; and one or more lipoic acid compounds in an amount from about 200 mg to about 400 mg.

In yet another embodiment of the disclosure, the Nutraceutical composition comprises one or more creatine compounds in an amount from about 4500 mg to about 5500 mg; one or more vitamin E compounds in an amount from about 50 mg to about 150 mg; one or more magnesium compounds in an amount from about 300 mg to about 500 mg; one or more zinc compounds in an amount from about 12 mg to about 18 mg; one or more DHA compounds in an amount from about 900 mg to about 1100 mg; and one or more lipoic acid compounds in an amount from about 250 mg to about 350 mg. The combination of Nutraceuticals may provide unexpected and greater than additive effects in the prophylactic treatment of SRTBI.

As previously mentioned, the Nutraceutical composition can be taken prophylactically. For example, the Nutraceutical composition can be taken as a daily dietary supplement. In some embodiments, the daily supplementation with the Nutraceutical composition can comprise taking the Nutraceutical composition each day for at least one week prior to participating in sports or other contact related activities. In other embodiments, daily supplementation with the Nutraceutical composition can comprise taking the Nutraceutical composition each day for at least five days prior to participating in sports or other contact related activities. In yet other embodiments, daily supplementation with the Nutraceutical composition can comprise taking the Nutraceutical each day for at least three days prior to participating in sports or other contact related activities. Daily supplementation with the Nutraceutical composition can further comprise taking the Nutraceutical composition each day for the duration of the time in which the contact related activities are expected to occur (e.g., for the length of the sports season, for the duration of training, etc.).

In some embodiments, the Nutraceutical composition may further optionally comprise an agent to promote the body's uptake of the Nutraceutical composition, such as Bioperine. The Nutraceutical composition can be used with other treatments, including, without limitation, pro-drugs and pharmaceutical derivatives, for administering co-therapies.

Various delivery systems can be used to administer the compounds or compositions disclosed herein, including, for example, encapsulation in emulsions, microparticles, and microcapsules. The required dosage can be administered as a single unit or in a sustained release form. In one embodiment, the Nutraceutical composition is administered in oral liquid dosage form including one or more agent, vehicle, or carrier and optional ingredients such as emulsifiers, colorants, flavorings, and other Nutraceuticals.

The Nutraceutical composition can be administered in solid dosage form. Solid dosage forms can include, but are not limited to, tablets, pills, powders, granules, and gels. In such solid dosage forms, the active compounds can be added to at least one inert diluent such as water, sucrose, lactose starch, or any combination thereof. Injectable preparations, for example, sterile injectable aqueous or oleaginous suspensions can be formulated according to the known art using suitable dispersing agents, wetting agents and/or suspending agents.

Different Nutraceutical compositions may also be administered by different delivery methods. For example, vitamin E may be administered orally in the form of a tablet, caplet, or gelcap, whereas creatine may be administered orally in liquid form. When the composition disclosed herein is in the form of multiple oral solid dosage forms, the doses can be administered as close in time as possible, or they can be administered hours apart. When the composition disclosed herein is administered as an oral liquid dosage form, the total volume of the dosage will be between 250 and approximately 1000 milliliters, approximately. The Nutraceutical composition can be administered by any available method of delivery.

The following examples are illustrative of certain embodiments disclosed herein and are not intended to be limiting in any way.

### Example 1

A study was performed to test the effectiveness of the Nutraceutical compositions disclosed herein. Two groups were used in this study, a Control Group and a Treatment Group. The Control Group consisted of Rugby players not given the Nutraceutical composition. The Treatment Group consisted of Rugby players taking a daily supplement of the Nutraceutical composition identified in the Table below, mixed with water or other non-acidic liquid and ingested orally. The players in the Treatment Group were given the supplement for the five days before their first game, and continued ingesting a daily supplement throughout the season.

| Ingredient | Amount | |
|---|---|---|
| Zinc Lactate Dihydrate | 15 | g |
| Magnesium Citrate Tribasic | 25 | g |
| Anhydrous | | |
| Creatine Monohydrate | 5000 | mg |
| Vitamin E | | |
| Tocotrienols | 50 | mg |
| Mixed Tocopherols | 25 | mg |
| Alpha Lipoic Acid | 250 | mg |
| DHA | 400 | mg |
| Uptake and/or Binding Aids | | |
| Bioperine | 25 | mg |
| Potassium | 21.4 | mg |
| Sugars | 8.7 | g |

Because of the difficulty in grading SRTBI or assessing it in the field, the effectiveness of the Nutraceutical composition was measured by the length of time (in days) required to return to play (RTP) after a player suffered a SRTBI. RTP protocols provide a way to measure the outcome of a SRTBI by measuring the length of time that a player is affected by the TBI symptoms. The stepwise return to play protocol begins once the player is symptom free according to the Sport Concussion Assessment Tool, 3rd Edition (SCAT3). Once the player no longer reports any TBI symptoms he/she is allowed to progressively engage in increasingly more strenuous physical activity. If any of the activities result in the resumption or reemergence of symptoms, then the player returns to the beginning of the stepwise progression. Used in this way, the amount of time it takes a players to return to play after a SRTBI offers a good measure of the outcome of the TBI. Cognitive testing can also be a part of the return to play protocol. All the participants were given a baseline cognitive skills test before the season and a retest was used in making RTP decisions in both the Control and Treatment Groups.

The results of the study showed that the average RTP for players in the Treatment Group was approximately 6 days. In contrast, the average RTP for players in the Control Group was approximately 10 days. While various factors can impact the results (e.g., the severity of the SRTBI, a player's susceptibility to SRTBI, etc.), these results indicate that a neuroprotective regime using the Nutraceutical composition disclosed herein is effective in ameliorating the effects of secondary injury associated with SRTBI.

It will be appreciated that reference throughout this specification to “an embodiment” or “the embodiment” means that a particular feature, structure or characteristic described in connection with that embodiment is included in at least one embodiment. Thus, the quoted phrases, or variations thereof, as recited throughout this specification are not necessarily all referring to the same embodiment.

Similarly, it should be appreciated that in the above description of embodiments, various features are sometimes grouped together in a single embodiment or description thereof for the purpose of streamlining the disclosure. This method of disclosure, however, is not to be interpreted as reflecting an intention that any claim require more features than those expressly recited in that claim. Rather, as the following claims reflect, inventive aspects lie in a combination of fewer than all features of any single foregoing disclosed embodiment. Thus, the claims following this Detailed Description are hereby expressly incorporated into this Detailed Description, with each claim standing on its own as a separate embodiment. This disclosure includes all permutations of the independent claims with their dependent claims.

The examples and embodiments disclosed herein are to be construed as merely illustrative and exemplary, and not a limitation of the scope of the present disclosure in any way. It will be apparent to those having skill with the aid of the present disclosure in the art that changes may be made to the details of the above-described embodiments without departing from the underlying principles of the disclosure herein. It is intended that the scope of the disclosure be defined by the claims appended hereto and their equivalents.

1. A nutraceutical composition, comprising:
one or more creatine compounds;
one or more vitamin E compounds;
one or more magnesium compounds;
one or more zinc compounds;
one or more DHA compounds; and
one or more lipoic acid compounds.

2. The composition of claim 1, wherein the amount of the one or more creatine compounds is from about 2500 mg to about 7000 mg; the amount of the one or more vitamin E compounds is from about 25 mg to about 400 mg; the amount of the one or more magnesium compounds is from about 100 mg to about 1000 mg; the amount of the one or more zinc compounds is from about 5 mg to about 50 mg; the amount of the one or more DHA compounds is from about 200 mg to about 2500 mg; and the amount of the one or more lipoic acid compounds is from about 100 mg to about 800 mg.

3. The composition of claim 1, wherein the amount of the one or more creatine compounds is from about 4000 mg to about 6000 mg; the amount of the one or more vitamin E compounds is from about 35 mg to about 200 mg; the amount of the one or more magnesium compounds is from about 250 mg to about 650 mg; the amount of the one or more zinc compounds is from about 10 mg to about 20 mg; the amount of the one or more DHA compounds is from about 800 mg to about 1200 mg; and the amount of the one or more lipoic acid compounds is from about 200 mg to about 400 mg.

4. The composition of claim 1, wherein the amount of the one or more creatine compounds is at least about 2500 mg; the amount of the one or more DHA compounds is at least about 200 mg; and the amount of the one or more lipoic acid compounds is at least about 100 mg.

5. The composition of claim 1, further comprising Bioperine.

6. A method of treating a condition associated with traumatic brain injury, comprising administering to an individual an effective amount of a nutraceutical composition comprising one or more creatine compounds, one or more vitamin E compounds, one or more magnesium compounds, one or more zinc compounds, one or more DHA compounds, and one or more lipoic acid compounds.

7. The method of claim 6, wherein the amount of the one or more creatine compounds is from about 2500 mg to about 7000 mg; the amount of the one or more vitamin E compounds is from about 25 mg to about 400 mg; the amount of the one or more magnesium compounds is from about 100 mg to about 1000 mg; the amount of the one or more zinc compounds is from about 5 mg to about 50 mg; the amount of the one or more DHA compounds is from about 200 mg to about 2500 mg; and the amount of the one or more lipoic acid compounds is from about 100 mg to about 800 mg.

8. The method of claim 6, wherein the amount of the one or more creatine compounds is from about 4000 mg to about 6000 mg; the amount of the one or more vitamin E compounds is from about 35 mg to about 200 mg; the amount of the one or more magnesium compounds is from about 250 mg to about 650 mg; the amount of the one or more zinc compounds is from about 10 mg to about 20 mg; the amount of the one or more DHA compounds is from about 800 mg to about 1200 mg; and the amount of the one or more lipoic acid compounds is from about 200 mg to about 400 mg.

9. The method of claim 6, wherein the amount of the one or more creatine compounds is at least about 2500 mg; the amount of the one or more DHA compounds is at least about 200 mg; and the amount of the one or more lipoic acid compounds is at least about 100 mg.

10. The method of claim 6, further comprising Bioperine.

11. The method of claim 6, wherein the nutraceutical composition is administered prophylactically.

12. The method of claim 6, wherein the nutraceutical composition is administered in oral liquid dosage form.

13. The method of claim 6, wherein the nutraceutical composition is administered in the form of at least one of an emulsion, a microparticle, or a microcapsule.

14. The method of claim 6, wherein the nutraceutical composition is administered as a single unit.

15. The method of claim 6, wherein the nutraceutical composition is administered in solid dosage form.

16. The method of claim 6, wherein the nutraceutical composition is administered with at least one inert diluent.

17. The method of claim 6, wherein the nutraceutical composition is administered as an injectable composition.

18. The method of claim 6, wherein the nutraceutical composition is administered as a daily dietary supplement.

19. A method of prophylactically treating a traumatic brain injury, comprising administering a nutraceutical composition as a daily dietary supplement to an individual, the nutraceutical composition comprising an effective amount of one or more creatine compounds, one or more vitamin E compounds, one or more magnesium compounds, one or more zinc compounds, one or more DHA compounds, and one or more lipoic acid compounds.

20. The method of claim 19, wherein the amount of the one or more creatine compounds is at least about 2500 mg; the amount of the one or more DHA compounds is at least about 200 mg; and the amount of the one or more lipoic acid compounds is at least about 100 mg.
